SEQUENCE LISTING (41 sequences)

SEQ ID NO: 1 - protein, 21 aa, Artificial
  CXCL12
MNAKVVVVLVLVLTALCLSDG

SEQ ID NO: 2 - protein, 27 aa, Artificial
  CXCL12
MGSPMASLVATLLVLTISLTFVSQSTA

SEQ ID NO: 3 - protein, 26 aa, Artificial
  CXCL12
MGKMASLFATFLVVLVSLSLASESSA

SEQ ID NO: 4 - protein, 24 aa, Artificial
  CXCL12
MANKHMSLSLFIVLLGLSCSLASG

SEQ ID NO: 5 - protein, 30 aa, Artificial
  CXCL12
MGFVLFSQLPSFLLVSTLLLFLVISHSCRA

SEQ ID NO: 6 - protein, 63 aa, Artificial
  CXCL12
SYRCPCRFFESHVARANVKHLKILNTPNCALQIVARLKNNNRQVCIDPKLKWIQEYLEKALNK

SEQ ID NO: 7 - protein, 63 aa, Artificial
  CXCL12
AYRCPCRFFESHVARANVKHLKILNTPNCALQIVARLKNNNRQVCIDPKLKWIQEYLEKALNK

SEQ ID NO: 8 - protein, 68 aa, Artificial
  CXCL12
KPVSLAYRCPCRFFESHVARANVKHLKILNTPNCALQIVARLKNNNRQVCIDPKLKWIQEYLEKALNK

SEQ ID NO: 9 - protein, 156 aa, Artificial
  CXCL12
KPVSLSYRCPCRFFESHVARANVKHLKILNTPNCALQIVARLKNNNRQVCIDPKLKWIQEYLEKALNKGGGGSGGGGSGGGGSGGGGSKPVSLSYRCPCRFFESHVARANVKHLKILNTPNCALQIVARLKNNNRQVCIDPKLKWIQEYLEKALNK

SEQ ID NO: 10 - protein, 315 aa, Artificial
  CXCL12
KPVSLSYRCPCRFFESHVARANVKHLKILNTPNCALQIVARLKNNNRQVCIDPKLKWIQEYLEKALNKGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1008 bp, Artificial
  CXCL12
atgaacgctaaggtagtagtcgtcttagtgttggtactcactgctctttgcttgtctgacgggaagcccgtatctctctcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataaggggggtggagggagcggaggaggaggatcaggaggtggtggatctgagcctaagagctgcgataagacccatacttgtcctccatgtcctgctccagaacttcttggtggtcctagcgttttcctgttccctccaaagcctaaggatacccttatgatcagcaggactcctgaggttacctgcgttgtggttgatgtgtctcatgaggatccagaggttaagttcaactggtatgtggatggtgttgaggtgcacaacgctaagactaagcctagagaggaacagtacaacagcacctacagggtggtgtctgtgcttactgttcttcaccaggattggctgaacggtaaagagtacaagtgcaaggtgagcaacaaggctctgcctgctcctatcgaaaagaccatctctaaggctaagggtcagcctagggaacctcaagtttacacccttccaccttctagggatgagctgaccaagaatcaggtgtcccttacctgcttggtgaagggattctacccatccgatatcgctgttgagtgggagtctaatggacagcctgagaacaactacaagaccacccctcctgtgctggattccgatggttcattcttcctgtacagcaagctgactgtggataagtctagatggcagcagggtaacgtgttcagctgctctgttatgcatgaggctctgcacaatcactacacccagaagagtctgtctctgtcccctggtaag

SEQ ID NO: 12 - DNA, 530 bp, Artificial
  CXCL12
tgaacgctaaggtagtagtcgtcttagtgttggtactcactgctctttgcttgtctgacgggaagcccgtatctctctcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataagggcggtggtggctccgggggaggagggagtgggggtgggggtagtggtggaggtggctccaagcccgtatctctctcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 13 - DNA, 252 bp, Artificial
  CXCL12
atgaacgctaaggtagtagtcgtcttagtgttggtactcactgctctttgcttgtctgacgggtcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 14 - DNA, 252 bp, Artificial
  CXCL12
atgaacgctaaggtagtagtcgtcttagtgttggtactcactgctctttgcttgtctgacggggcttataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 15 - DNA, 267 bp, Artificial
  CXCL12
atgaacgctaaggtagtagtcgtcttagtgttggtactcactgctctttgcttgtctgacgggaagcccgtatctctcgcttataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 16 - DNA, 270 bp, Artificial
  CXCL12
atgggatctccaatggccagcttggtggcaacactgttagtactcaccattagtcttacttttgtttcacaatccactgcttcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 17 - DNA, 270 bp, Artificial
  CXCL12
atgggatctccaatggccagcttggtggcaacactgttagtactcaccattagtcttacttttgtttcacaatccactgctgcttataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 18 - DNA, 285 bp, Artificial
  CXCL12
atgggatctccaatggccagcttggtggcaacactgttagtactcaccattagtcttacttttgtttcacaatccactgctaagcccgtatctctcgcttataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 19 - DNA, 267 bp, Artificial
  CXCL12
atgaacgccaaagtagttgtagtgttggtgctcgtcttgacggcattatgtttgtctgatggtaaacccgtgtctttgtcctatcgatgtccgtgcagattctttgagagtcacgtcgccagggccaatgttaagcacctgaagatactcaacaccccgaactgcgctttgcaaatagtcgctaggttaaagaataacaatagacaggtgtgtatagacccaaaattgaaatggattcaagaatatctcgaaaaggctcttaataag

SEQ ID NO: 20 - DNA, 267 bp, Artificial
  CXCL12
atgaacgctaaggtagtagtcgtcttagtgttggtactcactgctctttgcttgtctgacgggaagcccgtatctctctcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 21 - DNA, 266 bp, Artificial
  CXCL12
tgaacgccaaggtcgttgttgttctcgtgttggtattgactgctctgtgtctgtccgatggaaaacctgtttcactttcatacagatgcccatgtaggttcttcgagtctcacgtcgcaagagctaatgtaaaacacttgaaaatcctcaatacccccaactgtgcacttcagatagtggccaggctgaaaaataataacagacaggtttgcattgatccaaagctgaagtggattcaagagtatttggagaaagcattgaacaag

SEQ ID NO: 22 - DNA, 267 bp, Artificial
  CXCL12
atgaatgccaaggtcgtagttgtcctggtcttagttttaaccgcattgtgcctttctgatggaaaacccgtgagtttatcatataggtgcccatgtaggttctttgagagtcatgtggcaagggcaaatgtaaaacatctcaaaattttaaacaccccaaactgtgctttacaaatcgtggcacgattgaaaaataataaccgacaagtctgcatcgaccctaaattaaaatggatacaagagtatcttgagaaggcattgaacaag

SEQ ID NO: 23 - DNA, 285 bp, Artificial
  CXCL12
atgggatctccaatggccagcttggtggcaacactgttagtactcaccattagtcttacttttgtttcacaatccactgctaagcccgtatctctctcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 24 - DNA, 282 bp, Artificial
  CXCL12
atgggaaaaatggcttctctttttgctactttccttgttgtgttggttagtctttctctagctagtgagagtagtgctaagcccgtatctctctcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 25 - DNA, 276 bp, Artificial
  CXCL12
atggcaaataagcatatgtctctgtcactatttattgttctcttgggtctttcctgtagtttagctagcggaaagcccgtatctctctcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 26 - DNA, 294 bp, Artificial
  CXCL12
atgggtttcgttctcttctcccagctgccatcgttcttacttgtttcaactttactgctattcctggtaatttcacacagctgccgagcgaagcccgtatctctctcatataggtgtccgtgccgattttttgagtcccacgtagcacgagctaatgttaaacacctgaaaattctgaacacccctaactgcgcacttcaaatagttgcaaggcttaaaaacaataatcgtcaagtttgtattgatcccaaattaaaatggatacaagagtacctggagaaggctctgaataag

SEQ ID NO: 27 - DNA, 267 bp, Artificial
  CXCL12
atgaacgccaaagtagttgtagtgttggtgctcgtcttgacggcattatgtttgtctgatggtaaacccgtgtctttgtcctatcgatgtccgtgcagattctttgagagtcacgtcgccagggccaatgttaagcacctgaagatactcaacaccccgaactgcgcttgccaaatagtcgctaggttaaagaataacaatagacaggtgtgtatagacccaaaattgaaatggattcaagaatatctcgaaaagtgtcttaataag

SEQ ID NO: 28 - DNA, 267 bp, Artificial
  CXCL12
atgaacgctaaggtagtagtcgtcttagtgttggtactcactgctctttgcttgtctgacgggaagcccgtatctctctcatataggtgtccgtgccgattttttgagtcccacgtagcacgagctaatgttaaacacctgaaaattctgaacacccctaactgcgcatgccaaatagttgcaaggcttaaaaacaataatcgtcaagtttgtattgatcccaaattaaaatggatacaagagtacctggagaagtgtctgaataag

SEQ ID NO: 29 - DNA, 267 bp, Artificial
  CXCL12
atgaacgccaaggtcgttgttgttctcgtgttggtattgactgctctgtgtctgtccgatggaaaacctgtttcactttcatacagatgcccatgtaggttcttcgagtctcacgtcgcaagagctaatgtaaaacacttgaaaatcctcaatacccccaactgtgcatgccagatagtggccaggctgaaaaataataacagacaggtttgcattgatccaaagctgaagtggattcaagagtatttggagaaatgtttgaacaag

SEQ ID NO: 30 - DNA, 267 bp, Artificial
  CXCL12
atgaatgccaaggtcgtagttgtcctggtcttagttttaaccgcattgtgcctttctgatggaaaacccgtgagtttatcatataggtgcccatgtaggttctttgagagtcatgtggcaagggcaaatgtaaaacatctcaaaattttaaacaccccaaactgtgcttgccaaatcgtggcacgattgaaaaataataaccgacaagtctgcatcgaccctaaattaaaatggatacaagagtatcttgagaagtgtttgaacaag

SEQ ID NO: 31 - DNA, 285 bp, Artificial
  CXCL12
atgggatctccaatggccagcttggtggcaacactgttagtactcaccattagtcttacttttgtttcacaatccactgctaagcccgtatctctctcatataggtgtccgtgccgattttttgagtcccacgtagcacgagctaatgttaaacacctgaaaattctgaacacccctaactgcgcatgccaaatagttgcaaggcttaaaaacaataatcgtcaagtttgtattgatcccaaattaaaatggatacaagagtacctggagaagtgtctgaataag

SEQ ID NO: 32 - DNA, 282 bp, Artificial
  CXCL12
atgggaaaaatggcttctctttttgctactttccttgttgtgttggttagtctttctctagctagtgagagtagtgctaagcccgtatctctctcatataggtgtccgtgccgattttttgagtcccacgtagcacgagctaatgttaaacacctgaaaattctgaacacccctaactgcgcatgccaaatagttgcaaggcttaaaaacaataatcgtcaagtttgtattgatcccaaattaaaatggatacaagagtacctggagaagtgtctgaataag

SEQ ID NO: 33 - DNA, 276 bp, Artificial
  CXCL12
atggcaaataagcatatgtctctgtcactatttattgttctcttgggtctttcctgtagtttagctagcggaaagcccgtatctctctcatataggtgtccgtgccgattttttgagtcccacgtagcacgagctaatgttaaacacctgaaaattctgaacacccctaactgcgcatgccaaatagttgcaaggcttaaaaacaataatcgtcaagtttgtattgatcccaaattaaaatggatacaagagtacctggagaagtgtctgaataag

SEQ ID NO: 34 - DNA, 294 bp, Artificial
  CXCL12
atgggtttcgttctcttctcccagctgccatcgttcttacttgtttcaactttactgctattcctggtaatttcacacagctgccgagcgaagcccgtatctctctcatataggtgtccgtgccgattttttgagtcccacgtagcacgagctaatgttaaacacctgaaaattctgaacacccctaactgcgcatgccaaatagttgcaaggcttaaaaacaataatcgtcaagtttgtattgatcccaaattaaaatggatacaagagtacctggagaagtgtctgaataag

SEQ ID NO: 35 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 36 - protein, 68 aa, Homo sapiens
KPVSLSYRCPCRFFESHVARANVKHLKILNTPNCALQIVARLKNNNRQVCIDPKLKWIQEYLEKALNK

SEQ ID NO: 37 - protein, 84 aa, Artificial
  CXCL12
MNAKVVVVLVLVLTALCLSDGSYRCPCRFFESHVARANVKHLKILNTPNCALQIVARLKNNNRQVCIDPKLKWIQEYLEKALNK

SEQ ID NO: 38 - protein, 89 aa, Artificial
  CXCL12
MNAKVVVVLVLVLTALCLSDGKPVSLAYRCPCRFFESHVARANVKHLKILNTPNCALQIVARLKNNNRQVCIDPKLKWIQEYLEKALNK

SEQ ID NO: 39 - protein, 84 aa, Artificial
  CXCL12
MNAKVVVVLVLVLTALCLSDGAYRCPCRFFESHVARANVKHLKILNTPNCALQIVARLKNNNRQVCIDPKLKWIQEYLEKALNK

SEQ ID NO: 40 - protein, 177 aa, Artificial
  CXCL12
MNAKVVVVLVLVLTALCLSDGKPVSLSYRCPCRFFESHVARANVKHLKILNTPNCALQIVARLKNNNRQVCIDPKLKWIQEYLEKALNKGGGGSGGGGSGGGGSGGGGSKPVSLSYRCPCRFFESHVARANVKHLKILNTPNCALQIVARLKNNNRQVCIDPKLKWIQEYLEKALNK

SEQ ID NO: 41 - protein, 336 aa, Artificial
  CXCL12
MNAKVVVVLVLVLTALCLSDGKPVSLSYRCPCRFFESHVARANVKHLKILNTPNCALQIVARLKNNNRQVCIDPKLKWIQEYLEKALNKGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2018/0222957 A1
(43) Pub. Date: Aug. 9, 2018

(54) MODIFIED CXCL12 POLYPEPTIDES AND USES THEREOF

(71) Applicant: ViCapsys, Inc., Cumming, GA (US)

(72) Inventors: Stephen J. McCormack, Las Vegas, NV (US); Surendra J. Chavan, Lilburn, GA (US)

(21) Appl. No.: 15/887,467

(22) Filed: Feb. 2, 2018

Related U.S. Application Data
(60) Provisional application No. 62/454,428, filed on Feb. 3, 2017.

(51) Int. Cl. C07K 14/52

(52) U.S. Cl. CPC C07K 14/521; A61K 38/00

(57) ABSTRACT

The present invention relates to modified CXCL12 polypeptides, modified CXCL12 locked monomers, and CXCL12 locked dimers. The invention further relates to methods for inhibiting signaling through CXCR4 receptors using the polypeptides of the invention.

### RELATED APPLICATIONS

This application claims the benefit, under 35 U.S.C. § 119(e), of U.S. Provisional Application No. 62/454,428, filed Feb. 3, 2017, the entire contents of which are incorporated by reference herein.

### STATEMENT REGARDING ELECTRONIC FILING OF A SEQUENCE LISTING

A Sequence Listing in ASCII text format, submitted under 37 C.F.R. § 1.821, entitled 1416-2_ST25.txt, 29,292 bytes in size, generated on Feb. 1, 2018 and filed via EFS-Web, is provided in lieu of a paper copy. The Sequence Listing is incorporated herein by reference into the specification for its disclosures.

### FIELD OF THE INVENTION

The present invention relates to modified CXCL12 polypeptides, CXCL12 locked monomers, and CXCL12 locked dimers. The invention further relates to methods for activating signaling through CXCR4 receptors using the polypeptides of the invention.

### BACKGROUND OF THE INVENTION

CXCL12, formerly known as stromal cell-derived factor-1 (SDF-1), is a cytokine that is the natural ligand for CXCR4 and CXCR7 receptors. CXCL12 is expressed in many tissues and cell types and is strongly chemotactic for lymphocytes and other hematopoietic cells at lower concentrations. Expression of CXCL12 is associated with suppression of the immune response and has been observed in many cancers. The ability of CXCL12 to suppress the immune response has been used to prevent rejection of transplants and implantable devices (see US Publication No. 2016/0184234).

There is a need in the art for improved activators of CXCL12/CXCR4 and CXCL12/CXCR7 signaling.

### SUMMARY OF THE INVENTION

The present invention is based, in part, on the development of modified CXCL12 polypeptides, modified CXCL12 locked monomers, and modified CXCL12 locked dimers that may be used to activate CXCL12/CXCR4 and/or CXCL12/CXCR7 signaling pathways. The invention further relates to polynucleotides encoding the CXCL12 polypeptides of the invention that are optimized for expression of CXCL12 in heterologous host expression systems.

Accordingly, one aspect of the invention relates to a recombinant CXCL12 polypeptide, the polypeptide comprising a leader sequence operably linked to a CXCL12 polypeptide sequence, wherein 1 to 5 of the first consecutive amino acid residues of the CXCL12 polypeptide sequence are deleted relative to the wild-type CXCL12 sequence.

Another aspect of the invention relates to a recombinant CXCL12 polypeptide, the polypeptide comprising a leader sequence and a CXCL12 polypeptide sequence, wherein the sixth amino acid residue of the CXCL12 polypeptide sequence is substituted relative to the wild-type CXCL12 sequence.

A further aspect of the invention relates to a recombinant CXCL12 polypeptide, the polypeptide comprising, in order, a leader sequence, a first CXCL12 polypeptide sequence, a linker sequence, and a second CXCL12 polypeptide sequence.

An additional aspect of the invention relates to a recombinant CXCL12 polypeptide, the polypeptide comprising, in order, a leader sequence, a CXCL12 polypeptide sequence, and a Fc sequence.

Another aspect of the invention relates to a recombinant CXCL12 polypeptide, wherein 1 to 5 of the first consecutive amino acid residues of the CXCL12 polypeptide sequence are deleted relative to the wild-type CXCL12 sequence.

A further aspect of the invention relates to a recombinant CXCL12 polypeptide, wherein the sixth amino acid residue of the CXCL12 polypeptide sequence is substituted relative to the wild-type CXCL12 sequence.

An additional aspect of the invention relates to a recombinant CXCL12 polypeptide, the polypeptide comprising, in order, a first CXCL12 polypeptide sequence, a linker sequence, and a second CXCL12 polypeptide sequence.

Another aspect of the invention relates to a recombinant CXCL12 polypeptide, the polypeptide comprising, in order, a CXCL12 polypeptide sequence and a Fc sequence.

An additional aspect of the invention relates to a recombinant CXCL12 locked monomer polypeptide, wherein at least one cysteine is substituted relative to the wild-type CXCL12 sequence, such that the polypeptide is unable to form a disulfide bond with another CXCL12 monomer, and wherein 1 to 5 of the first consecutive amino acid residues of the CXCL12 polypeptide sequence deleted relative to the wild-type CXCL12 sequence.

A further aspect of the invention relates to a CXCL12 locked dimer polypeptide, wherein the dimer comprises two monomers locked together, and wherein at least one monomer has 1 to 5 of the first consecutive amino acid residues of the CXCL12 polypeptide sequence deleted relative to the wild-type CXCL12 sequence.

An additional aspect of the invention relates to a CXCL12 locked dimer polypeptide, wherein the dimer comprises two monomers locked together, and wherein at least one monomer has the sixth amino acid residue substituted relative to the wild-type CXCL12 sequence.

Another aspect of the invention relates to a prokaryotic or eukaryotic cell expressing the recombinant CXCL12 polypeptide, CXCL12 locked monomer, or CXCL12 locked dimer polypeptide of the invention.

A further aspect of the invention relates to a prokaryotic or eukaryotic cell culture medium comprising the recombinant CXCL12 polypeptide, CXCL12 locked monomer, or CXCL12 locked dimer polypeptide of the invention.

An additional aspect of the invention relates to a polynucleotide encoding the recombinant CXCL12 polypeptide of the invention, and an expression vector and host cell comprising the polynucleotide.

Another aspect of the invention relates to a method for producing the recombinant CXCL12 polypeptide, CXCL12 locked monomer, or CXCL12 locked dimer polypeptide of the invention, the method comprising: introducing the expression vector of the invention into a host cell to provide a recombinant cell; and culturing the cell in cell culture medium under conditions such that the recombinant CXCL12 polypeptide, CXCL12 locked monomer, or CXCL12 locked dimer polypeptide is expressed by the recombinant cell and secreted into the cell culture medium.

An additional aspect of the invention relates to a activating a CXCR4 receptor and/or CXCR7 receptor, comprising contacting the receptor with the recombinant CXCL12 polypeptide, CXCL12 locked monomer, or CXCL12 locked dimer polypeptide of the invention, thereby activating the receptor.

A further aspect of the invention relates to a method of inhibiting an immune response in a subject, comprising administering to the subject the recombinant CXCL12 polypeptide, CXCL12 locked monomer, or CXCL12 locked dimer polypeptide of the invention, thereby inhibiting an immune response.

Another aspect of the invention relates to a method of inhibiting an inflammatory response in a subject, comprising administering to the subject the recombinant CXCL12 polypeptide, CXCL12 locked monomer, or CXCL12 locked dimer polypeptide of the invention, thereby inhibiting an inflammatory response.

An additional aspect of the invention relates to a method of treating a CXCL12-responsive disease in a subject in need thereof, comprising administering to the subject the recombinant CXCL12 polypeptide, CXCL12 lock monomer polypeptide, or CXCL12 locked dimer polypeptide of any the invention, thereby treating the CXCL12-responsive disease.

An additional aspect of the invention relates to a substrate comprising the recombinant CXCL12 polypeptide or the CXCL12 locked dimer polypeptide of the invention, wherein the recombinant CXCL12 polypeptide or the CXCL12 locked dimer polypeptide is in and/or on the substrate.

These and other aspects of the invention are set forth in more detail in the description of the invention below.

### BRIEF DESCRIPTION OF THE DRAWINGS

FIG. 1 shows the effect of wild-type CXCL12 on THP-1 cell migration.

FIG. 2 shows the effect of locked dimer CXCL12 on THP-1 cell migration.

FIG. 3 shows the effect of locked dimer CXCL12 on wild-type CXCL12 induced migration of THP-1 cell cells.

FIG. 4 shows the effect of locked monomer CXCL12 on THP-1 cell migration.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in receptor binding and or activation (e.g., to CXCR4) of at least about 50% or more as compared to the activity of a polypeptide consisting of the recited sequence. The term “materially altered,” as applied to polynucleotides of the invention, refers to an increase or decrease in ability to inhibit expression of a target mRNA of at least about 50% or more as compared to the expression level of a polynucleotide consisting of the recited sequence.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof as used with respect to a polypeptide and a receptor, refers to bringing the polypeptide and the receptor in sufficiently close proximity to each other for one to exert a biological effect on the other. In some embodiments, the term contact means binding of the polypeptide to the receptor.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

The term “fragment,” as applied to a peptide, will be understood to mean an amino acid sequence of reduced length relative to a reference peptide or amino acid sequence and comprising, consisting essentially of, and/or consisting of an amino acid sequence of contiguous amino acids identical to the reference peptide or amino acid sequence. Such a peptide fragment according to the invention may be, where appropriate, included in a larger polypeptide of which it is a constituent. In some embodiments, such fragments can comprise, consist essentially of, and/or consist of peptides having a length of at least about 5, 10, 15, 20, 25, 30, 35, 46. 50, 55, or 60 or more consecutive amino acids of a peptide or amino acid sequence according to the invention. In other embodiments, such fragments can comprise, consist essentially of, and/or consist of peptides having a length of less than about 60, 55, 50, 45, 40, 35, 30, 25, 20, 15, 10, or 5 or less consecutive amino acids of a peptide or amino acid sequence according to the invention.

As used herein, the terms “protein” and “polypeptide” are used interchangeably and encompass both peptides and proteins, unless indicated otherwise.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a peptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, etc.

As used herein, a “functional” polypeptide or “functional fragment” is one that substantially retains at least one biological activity normally associated with that polypeptide (e.g., binding to or activating CXCR4 and/or CXCR7). In particular embodiments, the “functional” polypeptide or “functional fragment” substantially retains all of the activities possessed by the unmodified polypeptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as receptor binding and activation can be measured using assays that are well known in the art and as described herein.

As used herein, the term “host cell” refers to a cell that is engineered to express the recombinant CXCL12 polypeptide. “Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example, those described in Current Protocols in Molecular Biology (Ausubel et al., eds. 1987) Supplement 30, section 7.7.18, Table 7.7.1. Preferably, default parameters are used for alignment. One alignment program is BLAST, using default parameters. Examples of the programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesviruses, baculoviruses, modified baculoviruses, papoviruses, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

The present invention is based, in part, on the development of modified CXCL12 polypeptides, modified CXCL12 locked monomers, and modified CXCL12 locked dimers that may be used to modulate, e.g., activate, CXCL12/CXCR4 and/or CXCL12/CXCR7 signaling pathways. The modified CXCL12 polypeptides, and polynucleotides encoding the polypeptides, have many advantages over wild-type CXCL12 sequences, including without limitation, increased efficiency in recombinant production of the polypeptide, altered CXCR4 and CXCR7 binding, altered biological activity, altered stability, altered post-translation modifications, and altered dimerization rate. In some embodiments, the modified CXCL12 polypeptides of the invention are produced at a higher level than wild-type CXCL12 polypeptide, e.g., at a level that is at least about 5%, 10%, 15%, 20%, 25%, 30%, 40%, 50%, 60%, 70%, 80%, 90%, or 100% or more higher than wild-type.

CXCL12, formerly known as stromal cell-derived factor-1 (SDF-1), is a cytokine that is the natural ligand for CXCR4 receptors. Six protein isoforms have been identified in human: alpha, beta, gamma, delta, epsilon and theta, produced by alternative splicing events. The human alpha isoform is produced as a 89 amino acid proprotein (SEQ ID NO: 35) comprising a 21 amino acid leader sequence which is cleaved to produce a mature 68 amino acid CXCL12 protein (SEQ ID NO:36).

A first aspect of the invention relates to a recombinant CXCL12 polypeptide, the polypeptide comprising a leader sequence operably linked to a CXCL12 polypeptide sequence, wherein 1 to 5 of the first consecutive amino acid residues of the CXCL12 polypeptide sequence are deleted relative to the wild-type CXCL12 sequence. As used herein, a “CXCL12 polypeptide” refers to the mature polypeptide after the leader sequence is cleaved. Thus, reference to the first five consecutive amino acid residues of the CXCL12 polypeptide sequence refers to the sequence KPVSL in human CXCL12 and the corresponding residues from CXCL12 of other species. In some embodiments, 1, 2, 3, 4, or 5 of the first consecutive amino acid residues of the CXCL12 polypeptide sequence are deleted. In some embodiments, the recombinant CXCL12 polypeptide comprises, consists essentially of, or consists of the amino acid sequence of SEQ ID NO:37.

Another aspect of the invention relates to a recombinant CXCL12 polypeptide, the polypeptide comprising a leader sequence and a CXCL12 polypeptide sequence, wherein the sixth amino acid residue of the CXCL12 polypeptide sequence is substituted relative to the wild-type CXCL12 sequence. The sixth amino acid residue in human CXCL12 is a serine. The residue may be substituted with a conservative substitution. In some embodiments, the residue is substituted with alanine. In some embodiments, the recombinant CXCL12 polypeptide comprises, consists essentially of, or consists of the amino acid sequence of SEQ ID NO:38.

In some embodiments, the deletion of 1 to 5 of the first consecutive amino acid residues of the CXCL12 polypeptide sequence is combined with the substitution of the sixth residue. Thus, the modified CXCL12 polypeptide may have 1, 2, 3, 4, or 5 of the first consecutive amino acid residues deleted in combination with substitution of the sixth residue, e.g., to alanine. In some embodiments, the recombinant CXCL12 polypeptide comprises, consists essentially of, or consists of the amino acid sequence of SEQ ID NO:39.

Conservative amino acid substitutions may be based on any characteristic known in the art, including the relative similarity or differences of the amino acid side-chain substituents, for example, their hydrophobicity, hydrophilicity, charge, size, and the like.

In identifying amino acid sequences encoding peptides other than those specifically disclosed herein, the hydropathic index of amino acids may be considered. The importance of the hydropathic amino acid index in conferring interactive biologic function on a protein is generally understood in the art (see, Kyte and Doolittle, J. Mol. Biol. 157:105 (1982); incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, id), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Accordingly, the hydropathic index of the amino acid (or amino acid sequence) may be considered when modifying the peptides specifically disclosed herein.

It is also understood in the art that the substitution of amino acids can be made on the basis of hydrophilicity. U.S. Pat. No. 4,554,101 (incorporated herein by reference in its entirety) states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±I); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Thus, the hydrophilicity of the amino acid (or amino acid sequence) may be considered when identifying additional peptides beyond those specifically disclosed herein.

A further aspect of the invention relates to a recombinant CXCL12 polypeptide, the polypeptide comprising, in order, a leader sequence, a first CXCL12 polypeptide sequence, a linker sequence, and a second CXCL12 polypeptide sequence. The linker may be any amino acid sequence that provides a suitable length and/or flexibility. The linker may have a length of 1, 2, 3, 4, 5, 6, 7, 8, 9, 10, 15, or 20 amino acids or more or may have a length of less than 2, 3, 4, 5, 6, 7, 8, 9, 10, 15, or 20 amino acids. In one embodiment, the linker sequence is a repeating sequence, e.g., a repeat of G₄S, e.g., (G₄S)₃. In some embodiments, the first and second CXCL12 polypeptide sequence may be the same or different. In some embodiments, the first and/or second CXCL12 polypeptide sequence is the CXCL12 polypeptide sequence described above, e.g., having 1, 2, 3, 4, or 5 of the first consecutive amino acid residues deleted and/or a substitution of the sixth residue, e.g., to alanine. In some embodiments, the recombinant CXCL12 polypeptide comprises, consists essentially of, or consists of the amino acid sequence of SEQ ID NO:40.

An additional aspect of the invention relates to a recombinant CXCL12 polypeptide, the polypeptide comprising, in order, a leader sequence, a CXCL12 polypeptide sequence, and a Fc sequence. The Fc region of an antibody is the tail region that interacts with the Fc receptor of the cell surface. Fc sequences are well known in the art. The recombinant CXCL12 polypeptide may further comprise a linker between the CXL12 sequence and the Fc sequence. In one embodiment, the linker sequence is a repeating sequence, e.g., a repeat of G₄S, e.g., (G₄S)₃. In some embodiments, the recombinant CXCL12 polypeptide comprises, consists essentially of, or consists of the amino acid sequence of SEQ ID NO:41.

In some embodiments, any of the CXCL12 polypeptide sequences of the invention may be modified to provide desirable effects. The modification may be any combination of additions, deletions, and substitutions to the amino acid sequence. In some embodiments, the CXCL12 polypeptide may be truncated, e.g., from the N-terminus and/or C-terminus. The truncation(s) may be of 1, 2, 3, 4, 5, 6, 7, 8, 9, 10, 15, or 20 or more amino acids. In some embodiments, the truncation(s) may be less than 2, 3, 4, 5, 6, 7, 8, 9, 10, 15, or 20 amino acids. In some embodiments, the CXCL12 polypeptide sequences of the invention may further comprise a substitution of one or more amino acid residues, e.g., 1, 2, 3, 4, 5, 6, 7, 8, 9, 10, 15, or 20 or more amino acid residues, e.g., less than 2, 3, 4, 5, 6, 7, 8, 9, 10, 15, or 20 amino acid residues.

In certain embodiments, any of the CXCL12 polypeptide sequences of the invention may further comprise a substitution of one or more residues to cysteine, e.g., in order to promote the formation of disulfide bonds between monomers, e.g., to form locked dimers. In some embodiments, one or two residues are substituted with cysteine. In some embodiments, residues 36 (leucine) and 65 (alanine) (numbering with respect to the human CXCL12 sequence) are substituted with cysteine. In some embodiments, the locked dimer does not comprise L36C and/or A65C.

In certain embodiments, any of the CXCL12 polypeptide sequences of the invention may further comprise a substitution of one or more residues, e.g., cysteine residues, e.g., in order to reduce the formation of disulfide bonds between monomers, e.g., to create locked monomers. In some embodiments, one or two cysteine residues are substituted with a different amino acid. In some embodiments, residues 55 and 58 (numbering with respect to the human CXCL12 sequence) are substituted with a different amino acid. In some embodiments, the locked monomer does not comprise C55L and/or C58I.

The CXCL12 polypeptide sequence of the invention may be a modification of any isoform of CXCL12. In some embodiments, the CXCL12 polypeptide sequence is from CXCL12-α. In other embodiments, the CXCL12 polypeptide sequence is from CXCL12-β. When the polypeptide of the invention comprises more than one CXCL12 polypeptide sequence, the sequences may be from the same isoform (e.g., two CXCL12-α isoforms) or different isoforms (e.g., one CXCL12-α and one CXCL12-β).

In some embodiments, the CXCL12 polypeptide sequence is a mammalian CXCL12 polypeptide sequence. In some embodiments, the CXCL12 polypeptide sequence is a human CXCL12 polypeptide sequence.

The leader sequence in the recombinant CXCL12 polypeptide of the invention may be any suitable leader sequence that allows the polypeptide to be secreted from the cell. In some embodiments, the leader sequence is a wild-type CXCL12 leader sequence. The human CXCL12 leader sequence has the amino acid sequence of SEQ ID NO:1.

In some embodiments, the leader sequence is a heterologous leader sequence, e.g., from a plant protein, e.g., from Arabidopsis extensin, Nicotiana extensin, barley alpha amylase, or PR1A. In some embodiments, the leader sequence comprises the amino acid sequence of any one of SEQ ID NOS:2-5.

Arabidopsis extensin (SEQ ID NO:2)
Nicotiana extensin (SEQ ID NO:3)
barley alpha amylase (SEQ ID NO:4)

### PR1A (SEQ ID NO:5)

A further aspect of the invention relates to a mature CXCL12 polypeptide without the leader sequence. Thus, the invention relates to a recombinant CXCL12 polypeptide, wherein 1 to 5 of the first consecutive amino acid residues of the CXCL12 polypeptide sequence are deleted relative to the wild-type CXCL12 sequence, e.g., 1, 2, 3, 4, or 5 of the first consecutive amino acid residues are deleted. In one embodiment, the recombinant CXCL12 polypeptide comprises, consists essentially of, or consists of the amino acid sequence of SEQ ID NO:6.

In some embodiments, the recombinant CXCL12 polypeptide, in addition to the deletion of 1 to 5 of the first consecutive amino acid residues, further comprises a substitution of the sixth amino acid residue of the CXCL12 polypeptide sequence relative to the wild-type CXCL12 sequence, e.g., a conservative substitution, e.g., to alanine. In some embodiments, the recombinant CXCL12 polypeptide comprises, consists essentially of, or consists of the amino acid sequence of SEQ ID NO:7.

Another aspect of the invention relates to a recombinant CXCL12 polypeptide, wherein the sixth amino acid residue of the CXCL12 polypeptide sequence is substituted relative to the wild-type CXCL12 sequence, e.g., a conservative substitution, e.g., substituted with alanine. In some embodiments, the recombinant CXCL12 polypeptide comprises, consists essentially of, or consists of the amino acid sequence of SEQ ID NO:8.

An additional aspect of the invention relates to a recombinant CXCL12 polypeptide, the polypeptide comprising, in order, a first CXCL12 polypeptide sequence, a linker sequence, and a second CXCL12 polypeptide sequence. The linker may have a length of 1, 2, 3, 4, 5, 6, 7, 8, 9, 10, 15, or 20 amino acids or more or may have a length of less than 2, 3, 4, 5, 6, 7, 8, 9, 10, 15, or 20 amino acids. In one embodiment, the linker sequence is a repeating sequence, e.g., a repeat of G₄S, e.g., (G₄S)₃. In some embodiments, the first and/or second CXCL12 polypeptide sequence is the CXCL12 polypeptide sequence described above, e.g., having 1, 2, 3, 4, or 5 of the first consecutive amino acid residues deleted and/or a substitution of the sixth residue, e.g., to alanine. In some embodiments, the recombinant CXCL12 polypeptide comprises, consists essentially of, or consists of the amino acid sequence of SEQ ID NO:9.

Another aspect of the invention relates to a recombinant CXCL12 polypeptide, the polypeptide comprising, in order, a CXCL12 polypeptide sequence and a Fc sequence. The recombinant CXCL12 polypeptide may further comprise a linker between the CXL12 sequence and the Fc sequence. The linker may have a length of 1, 2, 3, 4, 5, 6, 7, 8, 9, 10, 15, or 20 amino acids or more or may have a length of less than 2, 3, 4, 5, 6, 7, 8, 9, 10, 15, or 20 amino acids. In one embodiment, the linker sequence is a repeating sequence, e.g., a repeat of G₄S, e.g., (G₄S)₃. In some embodiments, the recombinant CXCL12 polypeptide comprises, consists essentially of, or consists of the amino acid sequence of SEQ ID NO:10.

In some embodiments, any of the CXCL12 polypeptide sequences of the invention may be modified to provide desirable effects. The modification may be any combination of additions, deletions, and substitutions to the amino acid sequence. In some embodiments, the CXCL12 polypeptide may be truncated, e.g., from the N-terminus and/or C-terminus. The truncation(s) may be of 1, 2, 3, 4, 5, 6, 7, 8, 9, 10, 15, or 20 or more amino acids. In some embodiments, the truncation(s) may be less than 2, 3, 4, 5, 6, 7, 8, 9, 10, 15, or 20 amino acids. In some embodiments, the CXCL12 polypeptide sequences of the invention may further comprise a substitution of one or more amino acid residues, e.g., 1, 2, 3, 4, 5, 6, 7, 8, 9, 10, 15, or 20 or more amino acid residues, e.g., less than 2, 3, 4, 5, 6, 7, 8, 9, 10, 15, or 20 amino acid residues.

In certain embodiments, any of the CXCL12 polypeptide sequences of the invention may further comprise a substitution of one or more residues to cysteine, e.g., in order to promote the formation of disulfide bonds between monomers, e.g., to form locked dimers. In some embodiments, one or two residues are substituted with cysteine. In some embodiments, residues 36 (leucine) and 65 (alanine) (numbering with respect to the human CXCL12 sequence) are substituted with cysteine. In some embodiments, the locked dimer does not comprise 36C and/or 65C.

In certain embodiments, any of the CXCL12 polypeptide sequences of the invention may further comprise a substitution of one or more residues, e.g., cysteine residues, e.g., in order to reduce the formation of disulfide bonds between monomers, e.g., to create locked monomers. In some embodiments, one or two cysteine residues are substituted with a different amino acid. In some embodiments, residues 55 and 58 (numbering with respect to the human CXCL12 sequence) are substituted with a different amino acid. In some embodiments, the locked monomer does not comprise C55L and/or C58I.

The CXCL12 polypeptide sequence of the invention may be a modification of any isoform of CXCL12. In some embodiments, the CXCL12 polypeptide sequence is from CXCL12-α. In other embodiments, the CXCL12 polypeptide sequence is from CXCL12-β. When the polypeptide of the invention comprises more than one CXCL12 polypeptide sequence, the sequences may be from the same isoform (e.g., two CXCL12-α isoforms) or different isoforms (e.g., one CXCL12-α and one CXCL12-β).

In some embodiments, the CXCL12 polypeptide sequence is a mammalian CXCL12 polypeptide sequence. In some embodiments, the CXCL12 polypeptide sequence is a human CXCL12 polypeptide sequence.

A further aspect of the invention relates to a recombinant CXCL12 locked monomer polypeptide, wherein at least one cysteine is substituted relative to the wild-type CXCL12 sequence, such that the polypeptide is unable to form a disulfide bond with another CXCL12 monomer. As used herein, a “locked monomer polypeptide” is a CXCL12 polypeptide that preferentially does not form a dimer when present in a liquid. In some embodiments, when a locked monomer polypeptide is present in a liquid, less than 10% of the polypeptide is in the form of a dimer, e.g., less than 10%, 9%, 8%, 7%, 6%, 5%, 4%, 3%, 2%, or 1%.

In some embodiments, two cysteines are substituted relative to the wild-type CXCL12 sequence, e.g., with a conservative substitution, e.g., alanine. In certain embodiments, cysteine residues at positions 55 and 58 (numbering with respect to human CXCL12) are substituted with a different amino acid. In some embodiments, the locked monomer does not comprise C55L and/or C58I.

The CXCL12 polypeptide sequence of the invention may be a modification of any isoform of CXCL12. In some embodiments, the CXCL12 polypeptide sequence is from CXCL12-α. In other embodiments, the CXCL12 polypeptide sequence is from CXCL12-β. When the polypeptide of the invention comprises more than one CXCL12 polypeptide sequence, the sequences may be from the same isoform (e.g., two CXCL12-α isoforms) or different isoforms (e.g., one CXCL12-α and one CXCL12-β).

In some embodiments, the CXCL12 polypeptide sequence is a mammalian CXCL12 polypeptide sequence. In some embodiments, the CXCL12 polypeptide sequence is a human CXCL12 polypeptide sequence.

Another aspect of the invention relates to a CXCL12 locked dimer polypeptide, wherein the dimer comprises two monomers locked together, and wherein at least one monomer has 1 to 5 of the first consecutive amino acid residues of the CXCL12 polypeptide sequence deleted relative to the wild-type CXCL12 sequence. As used herein, a “locked dimer polypeptide” is a CXCL12 polypeptide that preferentially is in the form of a dimer when present in a liquid. In some embodiments, when a locked dimer polypeptide is present in a liquid, less than 10% of the polypeptide is in the form of a monomer, e.g., less than 10%, 9%, 8%, 7%, 6%, 5%, 4%, 3%, 2%, or 1%.

In some embodiments, 1, 2, 3, 4, or 5 consecutive amino acid residues of the CXCL12 polypeptide sequence are deleted relative to the wild-type CXCL12 sequence. In certain embodiments, at least one monomer has the sixth amino acid residue of the CXCL12 polypeptide sequence substituted relative to the wild-type CXCL12 sequence, e.g., substituted with alanine. The monomer with the sixth residue substituted may be the same or different from the monomer with the deletion.

A further aspect of the invention relates to a CXCL12 locked dimer polypeptide, wherein the dimer comprises two monomers locked together, and wherein at least one monomer has the sixth amino acid residue substituted relative to the wild-type CXCL12 sequence, e.g., substituted with alanine. In some embodiments, both monomers comprise the substitution.

The CXCL12 locked dimer polypeptide of the invention may be locked by substituting one or more amino acid residues in the monomers with cysteine. In some embodiments, two residues are substituted with cysteine. In certain embodiments, residues at positions 36 and 65 (numbering with respect to human CXCL12) are substituted with cysteine. In some embodiments, the locked dimer does not comprise L36C and/or A65C.

In certain embodiments, the polypeptide of the invention has at least 80% identity to any one of the sequences disclosed herein, e.g., at least 85%, 90%, 95%, 96%, 97%, 98%, 98%, or 99% identity.

In certain embodiments, the polypeptide of the invention can be modified for in vivo use, e.g., by the addition, at the amino- and/or carboxyl-terminal ends, of a blocking agent to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. For example, one or more non-naturally occurring amino acids, such as D-alanine, can be added to the termini. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Additionally, the peptide terminus can be modified, e.g., by acetylation of the N-terminus and/or amidation of the C-terminus. Likewise, the peptides can be covalently or noncovalently coupled to pharmaceutically acceptable “carrier” proteins prior to administration.

In one aspect, the invention is directed to a prokaryotic cell expressing a recombinant CXCL12 polypeptide as described herein. In one embodiment, the prokaryotic cell is Escherichia coli, Bacilus subtilis, Lactoccocus lactis, a Pseudomonas species, a Streptomyces species, a coryneform bacteria, or a halophilic bacteria.

In one aspect, the invention is directed to a eukaryotic cell expressing the recombinant CXCL12 polypeptide as described herein. In one embodiment, the eukaryotic cell is selected from the group consisting of a plant cell, a mammalian cell, a fungus cell, an insect cell, and a yeast cell. In one embodiment, the plant cell is an algae, a tobacco, C. roseus, N. benthamiana, or N. tabacum.

In one aspect, the invention is directed to a cell culture medium comprising the recombinant CXCL12 polypeptide as described herein. In one embodiment, the cell culture medium is a medium suitable for culture of prokaryotic cells. In one embodiment, the cell culture medium is a medium suitable for culture of eukaryotic cells.

Another aspect of the invention relates to a polynucleotide encoding the recombinant CXCL12 polypeptide of the invention. In some embodiments, the polynucleotide is operably linked to a polynucleotide encoding a leader sequence.

Those skilled in the art will appreciate that the isolated polynucleotides encoding the polypeptides of the invention will typically be associated with appropriate expression control sequences, e.g., transcription/translation control signals and polyadenylation signals.

It will further be appreciated that a variety of promoter/enhancer elements can be used depending on the level and tissue-specific expression desired. The promoter can be constitutive or inducible, depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest.

To illustrate, the polypeptide coding sequence can be operatively associated with a cytomegalovirus (CMV) major immediate-early promoter, an albumin promoter, an Elongation Factor 1-α (EF1-α) promoter, a PγK promoter, a MFG promoter, or a Rous sarcoma virus promoter.

Inducible promoter/enhancer elements include hormone-inducible and metal-inducible elements, and other promoters regulated by exogenously supplied compounds, including without limitation, the zinc-inducible metallothionein (MT) promoter; the dexamethasone (Dex)-inducible mouse mammary tumor virus (MMTV) promoter; the T7 polymerase promoter system (see WO 98/10088); the ecdysone insect promoter (No et al., Proc. Natl. Acad. Sci. USA 93:3346 (1996)); the tetracycline-repressible system (Gossen et al., Proc. Natl. Acad. Sci. USA 89:5547 (1992)); the tetracycline-inducible system (Gossen et al., Science 268:1766 (1995); see also Harvey et al., Curr. Opin. Chem. Biol. 2:512 (1998)); the RU486-inducible system (Wang et al., Nat. Biotech. 15:239 (1997); Wang et al., Gene Ther., 4:432 (1997)); and the rapamycin-inducible system (Magari et al., J. Clin. Invest. 100:2865 (1997)).

In some embodiments, the promoter is a promoter that is functional in plants, e.g., tobacco.

Moreover, specific initiation signals are generally required for efficient translation of inserted polypeptide coding sequences. These translational control sequences, which can include the ATG initiation codon and adjacent sequences, can be of a variety of origins, both natural and synthetic.

In some embodiments, the polynucleotide comprises, consists essentially of, or consists of a nucleotide sequence selected from one of SEQ ID NOS:11-18.

In certain embodiments, the polynucleotide is codon-optimized for expression in a host cell or organism. In some embodiments, the polynucleotide is codon-optimized for expression in a plant cell, e.g., Nicotiana benthamiana or Nicotiana tabacum.

In some embodiments, the polynucleotide comprises, consists essentially of, or consists of a nucleotide sequence selected from one of SEQ ID NOS:19-26.

In certain embodiments, the polynucleotide sequence is modified to encode a cysteine at positions 36 and 65 of the CXCL12 polypeptide (numbering with respect to human CXCL12). In some embodiments, the polynucleotide comprises, consists essentially of, or consists of a nucleotide sequence selected from one of SEQ ID NOS: 27-34.

In certain embodiments, the polynucleotide of the invention has at least 80% identity to any one of the sequences disclosed herein, e.g., at least 85%, 90%, 95%, 96%, 97%, 98%, 98%, or 99% identity.

Another aspect of the invention relates to an expression vector comprising the polynucleotide of the invention. Suitable expression vectors include, without limitation, plasmids and viral vectors.

For purposes of the invention, the regulatory regions (i.e., promoters, transcriptional regulatory regions, and translational termination regions) in the expression vector can be native/analogous to the organism or cell and/or the regulatory regions can be native/analogous to the other regulatory regions. Alternatively, the regulatory regions may be heterologous to the organism or cell and/or to each other (i.e., the regulatory regions). Thus, for example, a promoter can be heterologous when it is operably linked to a polynucleotide from a species different from the species from which the polynucleotide was derived. Alternatively, a promoter can also be heterologous to a selected nucleotide sequence if the promoter is from the same/analogous species from which the polynucleotide is derived, but one or both (i.e., promoter and polynucleotide) are substantially modified from their original form and/or genomic locus, or the promoter is not the native promoter for the operably linked polynucleotide.

In addition to the promoters described above, the expression vector also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences and polyadenylation signal sequences.

The expression vector also can optionally include a transcriptional and/or translational termination region (i.e., termination region) that is functional in the organism. A variety of transcriptional terminators are available for use in expression vectors and are responsible for the termination of transcription beyond the transgene and correct mRNA polyadenylation. The termination region may be native to the transcriptional initiation region, may be native to the operably linked nucleotide sequence of interest, may be native to the host, or may be derived from another source (i.e., foreign or heterologous to the promoter, the nucleotide sequence of interest, the host, or any combination thereof).

Regardless of the type of regulatory sequence(s) used, they can be operably linked to the nucleotide sequence of the CXCL12 polynucleotide. As used herein, “operably linked” means that elements of a nucleic acid construct such as an expression cassette are configured so as to perform their usual function. Thus, regulatory or control sequences (e.g., promoters) operably linked to a nucleotide sequence of interest are capable of effecting expression of the nucleotide sequence of interest. The control sequences need not be contiguous with the nucleotide sequence of interest, so long as they function to direct the expression thereof. Thus, for example, intervening untranslated, yet transcribed, sequences can be present between a promoter and a coding sequence, and the promoter sequence can still be considered “operably linked” to the coding sequence. A nucleotide sequence of the present invention can be operably linked to a regulatory sequence, thereby allowing its expression in a cell and/or subject.

The expression vector also can include a nucleotide sequence for a selectable marker, which can be used to select a transformed organism or cell. As used herein, “selectable marker” means a nucleic acid that when expressed imparts a distinct phenotype to the organism or cell expressing the marker and thus allows such transformed organisms or cells to be distinguished from those that do not have the marker. Such a nucleic acid may encode either a selectable or screenable marker, depending on whether the marker confers a trait that can be selected for by chemical means, such as by using a selective agent (e.g., an antibiotic or the like), or on whether the marker is simply a trait that one can identify through observation or testing, such as by screening. Of course, many examples of suitable selectable markers are known in the art and can be used in the expression vectors described herein.

Another aspect of the invention relates to a host cell comprising the polynucleotide or the expression vector of the invention. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, plant cells, and bacterial cells as described above and constructed using well known methods. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; DEAE-dextran; electroporation; or microinjection.

In one aspect, this invention is directed to a method for producing the recombinant CXCL12 polypeptide as described herein, the method comprising: introducing an expression vector comprising a nucleotide sequence coding for the recombinant CXCL12 polypeptide into a host cell to provide a recombinant cell; and culturing the cell in cell culture medium under conditions such that the recombinant CXCL12 polypeptide is expressed by the recombinant cell and secreted into the cell culture medium. In some embodiments, the method further comprises collecting the cell culture medium containing the recombinant CXCL12 polypeptide. In some embodiments, the method further comprises isolating or purifying the recombinant CXCL12 polypeptide from the collected medium.

The recombinant CXCL12 polypeptides, CXCL12 locked monomers, and CXCL12 locked dimer polypeptides of the invention are expected to have some or all of the biological activities of wild-type CXCL12 and may have one or more enhanced biological activities relative to wild-type CXCL12. Thus, the polypeptides of the invention may be used in the same manner as CXCL12 as is known in the art. Locked monomers and locked dimers of CXCL12 may be expected to have some distinct biological activities based on their effects on the CXCR4 and/or CXCR7 receptors. At lower concentrations (e.g., less than 100 nM), CXCL12 exists as a monomer. At higher concentrations, CXCL12 dimerizes, and when it binds to receptors it brings two receptors together. The downstream signaling from receptor monomers and receptor dimers is distinct. For example, activation of CXCR4 and CXCR7 monomers produces a chemoattractant effect on certain immune system cells while activation of CXCR4 and CXCR7 dimers produces a chemorepellant effect on those cells. The use of the CXCL12 locked monomers and locked dimers of the present invention advantageously provides one or the other signal in a more concentration-independent fashion by disrupting the normal concentration gradient of CXCL12 monomers and dimers.

One aspect of the invention relates to a method of activating a CXCR4 and/or CXCR7 receptor, comprising contacting the receptor with the recombinant CXCL12 polypeptide, CXCL12 locked monomer, or CXCL12 locked dimer polypeptide of the invention, thereby activating the receptor.

Another aspect of the invention relates to a method of inhibiting an immune response in a subject, comprising administering to the subject the recombinant CXCL12 polypeptide, CXCL12 locked monomer, or CXCL12 locked dimer polypeptide of the invention, thereby inhibiting an immune response. In some embodiments, the polypeptide is administered systemically. In some embodiments, the polypeptide is administered locally, and the immune response is inhibited locally, e.g., at the site of a transplant or implant (e.g., a medical device or tissue).

Another aspect of the invention relates to a method of inhibiting an inflammatory response in a subject, comprising administering to the subject the recombinant CXCL12 polypeptide, CXCL12 locked monomer, or CXCL12 locked dimer polypeptide of the invention, thereby inhibiting an inflammatory response. In some embodiments, the polypeptide is administered systemically. In some embodiments, the polypeptide is administered locally, and the inflammatory response is inhibited locally, e.g., at the site of a transplant or implant (e.g., a medical device or tissue).

Another aspect of the invention relates to a method of treating a CXCL12-responsive disease in a subject in need thereof, comprising administering to the subject the recombinant CXCL12 polypeptide, CXCL12 locked monomer, or CXCL12 locked dimer polypeptide of the invention, thereby treating the disease. As used herein a “CXCL12-responsive disease” is any disease, disorder, or condition or a symptom of any disease, disorder, or condition that improves from administration of CXCL12 to a subject having the disease, disorder, or condition. In some embodiments, the CXCL12-responsive disease, disorder, or condition is an inflammatory disease, an autoimmune disease, or a benign or malignant hyperproliferative disease (such as solid tumors and blood cancers). Examples of CXCL12-responsive diseases, disorders, and conditions include, without limitation, type 1 diabetes, type 2 diabetes, rheumatoid arthritis, osteoarthritis, multiple sclerosis, inflammatory bowel disease, tumors, blood cancers, wounds, surgical scars, keloids, fibrosis, adhesions, transplant rejection, and implant rejection (e.g., medical devices, hip and knee replacements, graft rejection, tendon repair).

A further aspect of the invention relates to a substrate comprising the recombinant CXCL12 polypeptide, CXCL12 locked monomer, or CXCL12 locked dimer polypeptide of the invention, wherein the recombinant CXCL12 polypeptide, CXCL12 locked monomer, or CXCL12 locked dimer polypeptide is in and/or on the substrate. In some embodiments, the substrate is a particle and the polypeptide is in the particle and/or on the surface of the particle. The particle (e.g., a microparticle or nanoparticle) may be composed of any material that is suitable for releasing the polypeptide of the invention and a desirable rate.

The particles may comprise a biocompatible material. The type of biocompatible material depends on the intended use. For example, the biocompatible material may be biodegradable or non-biodegradable. Without being bound by theory, it is believed that a biodegradable particle is preferred, for example, where the CXCL12 polypeptide of the invention is required at the site of implantation for a short period of time (e.g., hours to days or a week); where the particle cannot easily be removed from the implantation site; and/or where particles may cause damage or injury if left in place for a long period of time. In contrast, and without being bound by theory, it is believed that a non-biodegradable nanoparticle is preferred, for example, where the CXCL12 polypeptide is required at the site of implantation for a long period of time (e.g., weeks to months or longer), and/or the particle can be easily removed from the implantation site.

In one embodiment, the biocompatible material is a biocompatible polymer. The biocompatible polymer can be carbohydrate-based, protein-based, and/or synthetic, e.g., PLA. Biocompatable materials suitable for use in matrices include, but are not limited to, poly-dimethyl-siloxane (PDMS), poly-glycerolsebacate (PGS), polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, heparin, other extracellular matrix proteins, MATRIGEL®, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, nondegradable polyurethanes, polystyrenes, polyvinyl chloride, polyvinyl fluoride, polyvinyl imidazole, chlorosulphonated polyolefins, polyethylene oxide, polyvinyl alcohol, nylon silicon, poly(styrene-block-butadiene), polynorbomene, and hydrogels. Other suitable polymers can be obtained by reference to The Polymer Handbook, 3rd edition (Wiley, N.Y., 1989). Combinations of these polymers may also be used. In one embodiment, the biocompatible polymer is alginate or heparin. In one embodiment, the biocompatible polymer is not alginate.

In some embodiments, the particle is part of an implant or transplant. The polypeptide may be in the implant or transplant and/or on the surface of the implant or transplant. In some embodiments, the polypeptide is released from the implant or transplant after it is placed in a subject, thereby exerting a biological effect (e.g., reduced immune response) locally around the implant or transplant. In certain embodiments, the CXCL12 polypeptide is released at a rate that produces a local concentration that is effective to reduce the immune response at the location, e.g., a concentration of about 100 nM or less, e.g., 100, 90, 80, 70, 60, 50, 40, 30, 20, or 10 nM or less.

In one embodiment, the polypeptides of the invention are administered directly to a subject. Generally, the polypeptides of the invention will be suspended in a pharmaceutically-acceptable carrier (e.g., physiological saline) and administered orally or by intravenous infusion, or administered subcutaneously, intramuscularly, intrathecally, intraperitoneally, intrarectally, intravaginally, intranasally, intragastrically, intratracheally, or intrapulmonarily. In another embodiment, the intratracheal or intrapulmonary delivery can be accomplished using a standard nebulizer, jet nebulizer, wire mesh nebulizer, dry powder inhaler, or metered dose inhaler. They can be delivered directly to the site of the disease or disorder, such as lungs, kidney, or intestines. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of polypeptides available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-; 20-, 50-, 100-, 150-, or more fold). Encapsulation of the polypeptides in a suitable delivery vehicle (e.g., polymeric microparticles or implantable devices) may increase the efficiency of delivery, particularly for oral delivery.

According to certain embodiments, the polypeptides can be targeted to specific cells or tissues in vivo. Targeting delivery vehicles, including liposomes and targeted systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., Biochemistry 25:5500 (1986); Ho et al., J. Biol. Chem. 262:13979 (1987); Ho et al., J Biol. Chem. 262:13973 (1987); and U.S. Pat. No. 4,957,735 to Huang et al., each of which is incorporated herein by reference in its entirety).

As a further aspect, the invention provides pharmaceutical formulations and methods of administering the same to achieve any of the therapeutic effects (e.g., modulation of sodium absorption) discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The polypeptides of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the polypeptide (including the physiologically acceptable salts thereof) is typically admixed with, inter alia, an acceptable carrier. The carrier can be a solid or a liquid, or both, and is preferably formulated with the polypeptide as a unit-dose formulation, for example, a tablet, which can contain from 0.01 or 0.5% to 95% or 99% by weight of the polypeptide. One or more polypeptides can be incorporated in the formulations of the invention, which can be prepared by any of the well-known techniques of pharmacy.

A further aspect of the invention is a method of treating subjects in vivo, comprising administering to a subject a pharmaceutical composition comprising a polypeptide of the invention in a pharmaceutically acceptable carrier, wherein the pharmaceutical composition is administered in a therapeutically effective amount. Administration of the polypeptides of the present invention to a human subject or an animal in need thereof can be by any means known in the art for administering compounds.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into the brain for delivery to the central nervous system, into the pancreas, or into a tumor or the tissue surrounding a tumor). The most suitable route in any given case will depend on the nature and severity of the condition being treated and on the nature of the particular polypeptide which is being used.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be a solid, semi-solid, or liquid, e.g., in the form of a spray or a gel.

For oral administration, the polypeptide can be administered in solid dosage forms, such as capsules, tablets, and powders, or in liquid dosage forms, such as elixirs, syrups, and suspensions. polypeptides can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the polypeptide in a flavored base, usually sucrose and acacia or tragacanth; and pastilles comprising the polypeptide in an inert base such as gelatin and glycerin or sucrose and acacia.

Formulations of the present invention suitable for parenteral administration comprise sterile aqueous and non-aqueous injection solutions of the polypeptide, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a polypeptide of the invention, in a unit dosage form in a sealed container. The polypeptide or salt is provided in the form of a lyophilizate which is capable of being reconstituted with a suitable pharmaceutically acceptable carrier to form a liquid composition suitable for injection thereof into a subject. The unit dosage form typically comprises from about 1 mg to about 10 grams of the polypeptide or salt. When the polypeptide or salt is substantially water-insoluble, a sufficient amount of emulsifying agent which is pharmaceutically acceptable can be employed in sufficient quantity to emulsify the polypeptide or salt in an aqueous carrier. One such useful emulsifying agent is phosphatidyl choline.

Formulations suitable for rectal administration are preferably presented as unit dose suppositories. These can be prepared by admixing the polypeptide with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the polypeptides. Suitable formulations comprise citrate or bis/tris buffer (pH 6) or ethanol/water and contain from 0.1 to 0.2M of the compound.

The polypeptide can alternatively be formulated for nasal administration or otherwise administered to the lungs of a subject by any suitable means, e.g., administered by an aerosol suspension of respirable particles comprising the polypeptide, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the polypeptide can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the polypeptide can likewise be produced with any solid particulate medicament aerosol generator, by techniques known in the pharmaceutical art.

Alternatively, one can administer the polypeptide in a local rather than systemic manner, for example, in a depot or sustained-release formulation.

Further, the present invention provides liposomal formulations of the polypeptides disclosed herein and salts thereof. The technology for forming liposomal suspensions is well known in the art. When the polypeptide or salt thereof is an aqueous-soluble salt, using conventional liposome technology, the same can be incorporated into lipid vesicles. In such an instance, due to the water solubility of the polypeptide or salt, the polypeptide or salt will be substantially entrained within the hydrophilic center or core of the liposomes. The lipid layer employed can be of any conventional composition and can either contain cholesterol or can be cholesterol-free. When the polypeptide or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the polypeptides disclosed herein or salts thereof, can be lyophilized to produce a lyophilizate which can be reconstituted with a pharmaceutically acceptable carrier, such as water, to regenerate a liposomal suspension.

In the case of water-insoluble polypeptides, a pharmaceutical composition can be prepared containing the water-insoluble polypeptide, such as for example, in an aqueous base emulsion. In such an instance, the composition will contain a sufficient amount of pharmaceutically acceptable emulsifying agent to emulsify the desired amount of the polypeptide. Particularly useful emulsifying agents include phosphatidyl cholines and lecithin.

In particular embodiments, the polypeptide is administered to the subject in a therapeutically effective amount, as that term is defined above. Dosages of pharmaceutically active polypeptides can be determined by methods known in the art, see, e.g., Remington's Pharmaceutical Sciences (Maack Publishing Co., Easton, Pa.). The therapeutically effective dosage of any specific polypeptide will vary somewhat from polypeptide to polypeptide, and patient to patient, and will depend upon the condition of the patient and the route of delivery. As a general proposition, a dosage from about 0.1 to about 50 mg/kg will have therapeutic efficacy, with all weights being calculated based upon the weight of the polypeptide, including the cases where a salt is employed. Toxicity concerns at the higher level can restrict intravenous dosages to a lower level such as up to about 10 mg/kg, with all weights being calculated based upon the weight of the polypeptide, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the polypeptide for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults. In some embodiments, the subject is in need of the methods of the present invention, e.g., a subject having or suspected of having a CXCL12-responsive disease.

### Example 1

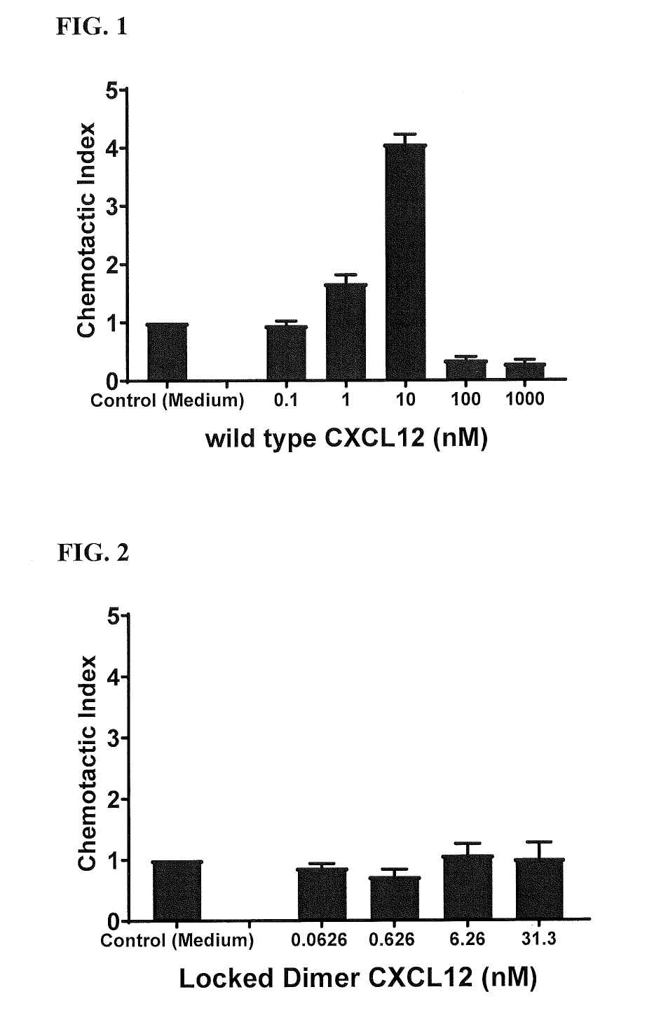

Dose dependent migration of THP-1 cells in response to wild type human CXCL12 was analyzed using a transwell assay. THP-1 cells were seeded in top chambers of a transwell and different compounds were added to the bottom wells at concentrations and combinations as indicated in FIG. 1. Cells were incubated at 37° C., 5% CO₂ for 3 hours. Following the incubation, cells that migrated to the bottom chamber were counted in quadruplicate using a hemocytometer. Chemotactic index was calculated as the ratio of cells counted in the treated groups vs. cells in the control group. An increase in cell migration was observed up to 10 nM CXCL12 and inhibition above 100 nM.

The effect of locked dimer CXCL12 on THP-1 migration was tested using a transwell assay. THP-1 cells were seeded in top chambers of a transwell and different compounds were added to bottom wells at concentrations and combinations as indicated in FIG. 2. Cells were incubated at 37° C., 5% CO₂ for 3 hours. Following the incubation, cells that migrated to the bottom chamber were counted in quadruplicate using a hemocytometer. The data show that locked dimer CXCL12 does not affect or induce migration of THP-1 cells.

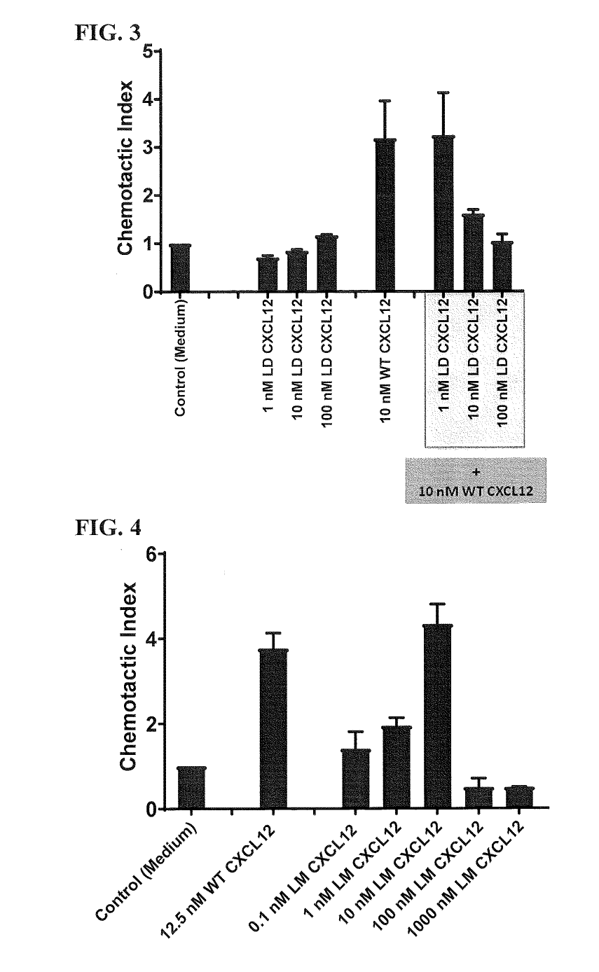

The ability of locked dimer (LD) CXCL12 to inhibit wild-type CXCL12-driven THP-1 migration was analyzed using a transwell assay. THP-1 cells were seeded in top chambers of a transwell and different compounds were added to bottom wells at concentrations and combinations as indicated in FIG. 3. Cells were incubated at 37° C., 5% CO₂ for 3 hours. Following the incubation, cells that migrated to the bottom chamber were counted in quadruplicate using a hemocytometer. The data shows that the locked dimer CXCL12 inhibited wild-type CXCL12 driven migration of THP-1 cells in a dose dependent manner.

The effect of locked monomer (LM) CXCL12 on THP-1 migration was analyzed using a transwell assay. THP-1 cells were seeded in top chambers of a transwell and different compounds were added to bottom wells at concentrations and combinations as indicated in FIG. 4. Cells were incubated at 37° C., 5% CO₂ for 3 hours. Following the incubation, cells that migrated to the bottom chamber were counted in quadruplicate using a hemocytometer. The data demonstrated a dose-dependent migration of THP-1 cells to locked monomer CXCL12 and inhibition of migration at the 100 nM dose.

The foregoing is illustrative of the present invention, and is not to be construed as limiting thereof. The invention is defined by the following claims, with equivalents of the claims to be included therein.

1. A recombinant CXCL12 polypeptide, the polypeptide comprising a leader sequence operably linked to a CXCL12 polypeptide sequence, wherein 1 to 5 of the first consecutive amino acid residues of the CXCL12 polypeptide sequence are deleted relative to the wild-type CXCL12 sequence.

2. (canceled)

3. The recombinant CXCL12 polypeptide of claim 1, wherein the sixth amino acid residue of the CXCL12 polypeptide sequence is substituted relative to the wild-type CXCL12 sequence.

4. (canceled)

5. A recombinant CXCL12 polypeptide, the polypeptide comprising a leader sequence operably linked to a CXCL12 polypeptide sequence, wherein the sixth amino acid residue of the CXCL12 polypeptide sequence is substituted relative to the wild-type CXCL12 sequence.

6. (canceled)

7. A recombinant CXCL12 polypeptide, the polypeptide comprising, in order, a leader sequence, a first CXCL12 polypeptide sequence, a linker sequence, and a second CXCL12 polypeptide sequence.

8-9. (canceled)

10. A recombinant CXCL12 polypeptide, the polypeptide comprising, in order, a leader sequence, a CXCL12 polypeptide sequence, and a Fc sequence.

11. The recombinant CXCL12 polypeptide of claim 1, wherein at least one residue in the CXCL12 polypeptide sequence is substituted with cysteine.

12. (canceled)

13. The recombinant CXCL12 polypeptide of claim 1, wherein at least one cysteine residue in the CXCL12 polypeptide sequence is substituted with a different amino acid.

14-56. (canceled)

57. A prokaryotic cell expressing the recombinant CXCL12 polypeptide of claim 1.

58. (canceled)

59. A eukaryotic cell expressing the recombinant CXCL12 polypeptide of claim 1.

60-61. (canceled)

62. A prokaryotic cell culture medium comprising the recombinant CXCL12 polypeptide of claim 1.

63. A eukaryotic cell culture medium comprising the recombinant CXCL12 polypeptide of claim 1.

64. A polynucleotide encoding the recombinant CXCL12 polypeptide of claim 1.

65. The polynucleotide of claim 64, codon-optimized for expression in a host cell.

66-72. (canceled)

73. The polynucleotide of claim 64, operably linked to a promoter.

74. An expression vector comprising the polynucleotide of claim 64.

75. A host cell comprising the polynucleotide of claim 1.

76. A method for producing the recombinant CXCL12 polypeptide of claim 1, the method comprising:
introducing an expression vector comprising a polynucleotide encoding the recombinant CXCL12 polypeptide of claim 1 into a host cell to provide a recombinant cell; and culturing the cell in cell culture medium under conditions such that the recombinant CXCL12 polypeptide is expressed by the recombinant cell and secreted into the cell culture medium.

77-78. (canceled)

79. A method of activating a CXCR4 receptor and/or CXCR7 receptor, comprising contacting the receptor with the recombinant CXCL12 polypeptide of claim 1, thereby activating the receptor.

80. A method of inhibiting an immune response in a subject, comprising administering to the subject the recombinant CXCL12 polypeptide of claim 1, thereby inhibiting an immune response.

81. (canceled)

82. A method of inhibiting an inflammatory response in a subject, comprising administering to the subject the recombinant CXCL12 polypeptide of claim 1, thereby inhibiting an inflammatory response.

83. (canceled)

84. A method of treating a CXCL12-responsive disease in a subject in need thereof, comprising administering to the subject the recombinant CXCL12 polypeptide of claim 1, thereby treating the CXCL12-responsive disease.

85. (canceled)

86. A substrate comprising the recombinant CXCL12 polypeptide of claim 1, wherein the recombinant CXCL12 polypeptide is in and/or on the substrate.

87. (canceled)
